(19) United States
(12) Patent Application Publication
(10) Pub. No.: US 2023/0365906 A1
(43) Pub. Date: Nov. 16, 2023

(54) RECIRCULATING BIOREACTOR

(71) Applicant: Stellular Bio, Inc., Watertown, MA (US)

(72) Inventor: Jonathan Thon, Cambridge, MA (US)

(73) Assignee: Stellular Bio, Inc., Watertown, MA (US)

(21) Appl. No.: 18/131,689

(22) Filed: Apr. 6, 2023

Related U.S. Application Data
(63) Continuation of application No. 16/490,711, filed on Sep. 3, 2019, now abandoned.
(60) Provisional application No. 62/468,008, filed on Mar. 7, 2017.

(51) Int. Cl. C12M 3/06; C12M 1/00; C12M 1/34

(52) U.S. Cl. CPC C12M 23/16; C12M 29/04; C12M 29/10; C12M 29/14; C12M 41/32

(57) ABSTRACT

A bioreactor including a bioreactor body, wherein the bioreactor body includes a first substrate and an opposing second substrate, a pathway extending through the bioreactor body and being formed by a first channel defined in the first substrate and an opposing second channel defined in the second substrate, a first inlet for introducing a first fluid flow to the first channel, a second inlet for introducing a second fluid flow to the second channel, a first outlet for permitting the first fluid flow to exit the first channel, a second outlet for permitting the second fluid flow to exit the second channel, a membrane disposed in the pathway between the first and second channels and having a plurality of pores sized to selectively capture, in the first channel, a biological source material and to permit biological products to be collected from the bioreactor.

### RELATED APPLICATIONS

This application is a continuation of U.S. patent application Ser. No. 16/490,711, filed on Sep. 3, 2019, which claims benefit of U.S. National Stage application, pursuant to 35 U.S.C. § 371, of PCT International Patent Application No. PCT/US2018/021354, filed Mar. 7, 2018, which claims priority to and the benefit of U.S. Provisional Application No. 62/468,008, filed Mar. 7, 2017, which is incorporated herein by reference in its entirety.

### STATEMENT OF GOVERNMENT SUPPORT

This invention was made with government support under 1R44HL131050-01 awarded by the National Institutes of Health (NIH). The government has certain rights in the invention.

### FIELD

The present disclosure generally relates to fluid systems and more particularly to bioreactors.

### BACKGROUND OF THE DISCLOSURE

In medical practice, various biological products can be used to treat various disorders, infections, malignancies, and traumas. Additionally, such biological products (e.g., plasma, platelets, white blood cells, red blood cells) can be used to replace depleted biological products within a patient. Production of such biological products has been attempted using various techniques such as production from various stem cells. Stem cells utilized have typically included embryonic stem cells, umbilical cord blood stem cells and induced pluripotent stem cells. Other stem cell sources have included stem cells found in bone marrow, fetal liver and peripheral blood. However, despite successful production of some biological products in the laboratory, many limitations remain to use in a clinical setting.

Therefore, in light of the above, there remains a need for efficient ways to produce clinically relevant yields of biological products that can meet growing clinical demands, and avoid the risks and costs associated with donor harvesting and storage.

### SUMMARY OF THE DISCLOSURE

The instant disclosure describes various bioreactor embodiments and methods of their use that include a number of features and capabilities aimed at generating clinically and commercially relevant biological products from biological source materials. In some embodiments, the system and method described herein may be used to generate high platelet yields usable for platelet infusion.

In some embodiments, a bioreactor is provided. The bioreactor includes one or more bioreactor bodies, wherein at least one bioreactor body includes a first channel and an opposing second channel. In operation, a biological source material capable of generating biological products is delivered to the first channel at a predetermined, adjustable flow rate, and the bioreactor further includes a membrane disposed between the first and second channels, the membrane including a plurality of pores sized to selectively capture, in the first channel, a biological source material and to permit the generated biological products to be collected from the first channel or pass through the membrane into the second channel.

In some embodiments, one or both of the first and second channels are sized and shaped to ensure a uniform distribution of the biological source material along the membrane. In some embodiments, the instant bioreactor allows decoupling of shear stress on the biological source material and the transmembrane pressure and varying the shear stress and pressure independently of one another. In some embodiments, the instant bioreactor is configured such that the shear stress and pressure can be controlled independently by adjusting the seeding density of the biological source product over the membrane to compensate for coupled properties of the shear stress and pressure. In some embodiments, the pore size is selected such that essentially all or all of the biological source material is trapped in the first channel, while all or essentially all of the biological product is allowed to pass into the second channel for collection. In some embodiments, the instant bioreactor is configured such that the flow media can be recirculated through the membrane and one or both of the first and second channels independently.

In some embodiments, the membrane is made from a material that allows the membrane to stretch under pressure or curve toward the first or second channel. In some embodiments, the bioreactor is made as a unitary unit. Alternatively, the bioreactor may be made of a first substrate having the first channel and a second substrate having the second channel, where the first substrate and the second substrate are bonded together, such as by adhesives, in a manner that prevents leaking of from the first channel or the second channel.

In some embodiments, the instant bioreactor allows control of shear and pressure through the channels in a tight range for most of the seeded cells (>80%). In some embodiments, the bioreactor shear profile can be regulated by adjusting the geometry of the channels. In some embodiments, the bioreactor allows recirculation during operation through any combination of its inlets and outlets. In some embodiments, the bioreactor can effectively retain biological materials which size is above the membrane pore size and allows passage biological products which size is below the membrane pore size.

In some embodiments, a bioreactor is provided. The bioreactor includes one or more bioreactor bodies, wherein at least one bioreactor body includes a first substrate and an opposing second substrate engaged with the first substrate. The bioreactor also includes a pathway extending through the bioreactor body and being formed by a first channel defined in the first substrate and an opposing second channel defined in the second substrate, the second channel being in alignment with the first channel. The bioreactor also includes a first inlet for introducing a first fluid flow to the first channel. The bioreactor also includes a second inlet for introducing a second fluid flow to the second channel. The bioreactor also includes a first outlet for permitting the first fluid flow to exit the first channel. The bioreactor also includes a second outlet for permitting the second fluid flow to exit the second channel. The bioreactor also includes a membrane disposed in the pathway between the first and second channels, the membrane including a plurality of pores, the pores being sized to selectively capture, in the first channel, a biological source material capable of generating biological products and to permit the generated biological products to be collected from the first channel or pass through the membrane into the second channel.

In some embodiments the pathway is a serpentine pathway. In some embodiments the biological source material includes one or more of cells including stem cells and/or intermediate and/or final product of stem cell differentiation such as hemogenic endothelia, hematopoietic progenitor cells, megakaryocytes, endothelial cells, leukocytes, erythrocytes bone marrow cells, blood cells, lung cells, cells comprising basement membranes, and/or small molecules including CCL5, CXCL12, CXCL10, SDF-1, FGF-4, S1PR1, RGDS, Methylcellulose, and extracellular matrix proteins including collagen, fibrinectin, fibrinogen, laminin, Matrigel, Flt-3, TPO, VEGF, PLL, IL3, 6, 9, 1b, vitronectin, or combinations thereof. In some embodiments the biological products include one or more of products of the biological source material, components of the biological source material, or combinations thereof. In some embodiments the biological source material includes megakaryocytes and the biological products include one or more of preplatelets, proplatelets, platelets or their component products. It should be noted that while the methods and devices of the present disclosure can be described in connection with megakaryocytes as the bioloigclal source material and preplatelets, proplatelets, platelets or their component products as the biological product, the instant methods and devices can also be used with other biological source materials generating other biological products. In some embodiments at least one of the first fluid flow and the second fluid flow includes a fluid media including one or more biological substances including one or more of cell culture media, growth factors, whole blood, plasma, platelet additive solutions, suspension media, saline, phosphate buffered saline, or combinations thereof.

In some embodiments the bioreactor also includes a third inlet for introducing the biological source material to the first channel. In some embodiments the bioreactor also includes a first recirculation line for recirculating the first fluid flow from the first outlet to the first inlet; and a second recirculation line for recirculating the second fluid flow from the first outlet to the second inlet. In some embodiments the bioreactor also includes a first pump for pumping the first fluid flow through the first recirculation line; and a second pump for pumping the second fluid flow through the second recirculation line. In some embodiments the bioreactor also includes a single pump for pumping the first fluid flow through the first recirculation line and for pumping the second fluid flow through the second recirculation line. In some embodiments the pores of the membrane are further sized to prevent the biological source materials and biological products from passing through the membrane. In some embodiments the bioreactor also includes a flow controller configured to control flow rates of the first and second fluid flows in the first and second channels to generate shear rates at the membrane within a predetermined range selected to facilitate production of biological products. In some embodiments the shear rates generated at the membrane are physiologically relevant and in a range approximately between 10 sec⁻¹ and 5000 sec⁻¹. In some embodiments the flow in at least one of the first channel or the second channel is one of peristaltic flow or laminar flow. In some embodiments the peristaltic flow is pulsatile with a physiologically relevant frequency between 40 and 120 pulses per minute. In some embodiments a shear rate generated at the membrane during the pulsatile peristaltic flow varies through a physiologically relevant range between 250 sec⁻¹ and 1800 sec⁻¹.

In some embodiments the first substrate is bonded to the second substrate. In some embodiments the membrane is bonded between the first and second substrates. In some embodiments a height of the first channel and a height of the second channel are sized to produce a uniform pressure drop across the membrane along the length of the pathway. In some embodiments a height of the first channel and a height of the second channel are sized to produce a uniform shear at the surface of the membrane along the length of the pathway. In some embodiments a taper angle formed between a surface of each channel and the membrane is in a range approximately between 0 and 5 degrees. In some embodiments the substrates comprise one or more of thermoplastics, glass, polymethyl methacrylate (PMMA), polydimethylsiloxane (PDMS), polycarbonate (PC), cyclic olefin copolymer (COC), cyclic olefin polymer (COP), polyvinyl chloride (PVC), coated polystyrene, coated glass, silk, hydrogels, or combinations thereof. In some embodiments the membrane comprises one or more of thermoplastics, glass, polymethyl methacrylate (PMMA), polydimethylsiloxane (PDMS), polycarbonate (PC), cyclic olefin copolymer (COC), cyclic olefin polymer (COP), polyvinyl chloride (PVC), coated polystyrene, coated glass, silk, hydrogels, or combinations thereof. In some embodiments the pores are sized in a range approximately between 0.1 micrometers and 50 micrometers. In some embodiments a pressure differential profile between the first channel and the second channel is substantially uniform over at least a portion of the membrane.

In some embodiments a method for generating biological products is provided. The method includes providing a bioreactor. The bioreactor includes at least one bioreactor body including a first substrate and an opposing second substrate engaged with the first substrate. The bioreactor also includes a pathway extending through the bioreactor body and being formed by a first channel defined in the first substrate and an opposing second channel defined in the second substrate, the second channel being in alignment with the first channel. The bioreactor also includes a membrane disposed in the pathway between the first and second channels, the membrane including a plurality of pores, the pores being sized to selectively capture, in the first channel, a biological source material capable of generating biological products and to permit the generated biological products to pass through the membrane into the second channel. The method also includes introducing the biological source material to the first channel to seed the bioreactor. The method also includes introducing a first fluid flow to the first channel via a first inlet of the bioreactor at a predetermined first flow rate and a second fluid flow to the second channel via a second inlet of the bioreactor at a predetermined second flow rate to generate the desired biological products. The method also includes harvesting the desired biological products from the bioreactor assembly.

In some embodiments the method also includes recirculating the first fluid flow from a first outlet of the first channel to the first inlet via a first recirculation line; and recirculating the second fluid flow from a second outlet of the second channel to the second inlet via a second recirculation line. In some embodiments the method also includes pumping, by a first pump, the first fluid flow through the first recirculation line; and pumping, by a second pump, the second fluid flow through the second recirculation line. In some embodiments the method also includes pumping, by a single pump, the first fluid flow through the first recirculation line and for pumping the second fluid flow through the second recirculation line. In some embodiments the method also includes generating the biological source material from bone marrow, peripheral blood, umbilical cord blood, fetal liver, yolk sack, spleen, or pluripotent stem cells. In some embodiments the step of introducing the biological source material further comprises flowing a fluid containing the biological source material into the first channel, wherein distribution of the biological source material along the membrane is mediated by the flow of the fluid containing the biological source material. In some embodiments the biological source material, when selectively captured by one of the pores, blocks the pore. In some embodiments the blockage of the pores by the selectively captured biological source material mediates fluid flow through the membrane. In some embodiments the method also includes monitoring a pressure drop across the membrane between the first channel and the second channel; and determining, from the pressure drop, a density of the biological source material within the introduced fluid containing the biological source material. In some embodiments the method also includes adjusting an introduced quantity of the introduced fluid containing the biological source material in response to the determined density.

In some embodiments, a bioreactor is provided. The bioreactor includes one or more bioreactor bodies, wherein at least one bioreactor body includes a first substrate and an opposing second substrate engaged with the first substrate. The bioreactor also includes a pathway extending through the bioreactor body and being formed by a first channel defined in the first substrate and an opposing second channel defined in the second substrate, the second channel being in alignment with the first channel. The bioreactor also includes a first inlet for introducing a first fluid flow to the first channel. The bioreactor also includes a second inlet for introducing a second fluid flow to the second channel. The bioreactor also includes a third inlet for delivering a biological source material capable of generating biological products to the first channel. The bioreactor also includes a first outlet for permitting the first fluid flow to exit the first channel. The bioreactor also includes a second outlet for permitting the second fluid flow to exit the second channel. The bioreactor also includes a membrane disposed in the pathway between the first and second channels, the membrane including a plurality of pores, the pores being sized to selectively capture, in the first channel, the biological source material and to permit the generated biological products to be collected from the first channel or pass through the membrane into the second channel.

The foregoing and other aspects and advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is an illustration showing in vivo platelet production in bone marrow.

FIG. 2 is a block diagram illustrating a system for producing biological products, in accordance with various embodiments.

FIGS. 3A and 3B are perspective and top views showing an embodiment of a bioreactor, in accordance with various embodiments.

FIG. 3C is a cross-sectional front view of the bioreactor in accordance with various embodiments.

FIG. 3D is a cross-sectional side view of the bioreactor in accordance with various embodiments.

FIGS. 3E and 3F are detail views of the side view of FIG. 3D of the bioreactor in accordance with various embodiments.

FIGS. 4A and 4B are cross-sectional side views illustrating a resting position and a stretched position of a flexible membrane of a bioreactor in accordance with various embodiments.

FIG. 5 is a schematic showing a recirculating bioreactor in accordance with various embodiments.

FIG. 6 is a cross-sectional view of a port having a bubble trap in accordance with various embodiments.

FIG. 7A is an image showing megakaryocyte distribution along a section of a bioreactor channel in accordance with various embodiments.

FIG. 7B is an image showing megakaryocyte distribution at various stations along a bioreactor channel in accordance with various embodiments.

FIGS. 8A and 8B are functional flow diagrams illustrating a pressure wave method for seeding a bioreactor in accordance with various embodiments.

FIG. 9 is a functional flow diagram illustrating a direct infusion method for seeding a bioreactor in accordance with various embodiments.

FIG. 10A and FIG. 10B are flow cytometry plots showing a mixed population of large nucleated cells and platelet sized particles prior to seeding the cells (FIG. 10A) and post seeding the cells (FIG. 10B) in the bioreactor.

FIGS. 11A-11C illustrate tablet, stacked tablet, and industrial bioreactors formed from a modular, scalable bioreactor system in accordance with various embodiments.

FIG. 12 is a cross-sectional view showing an embodiment of a single reservoir vessel bioreactor, in accordance with various embodiments.

FIG. 13 is flow diagrams illustrating a method for seeding a bioreactor in accordance with various embodiments.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE DISCLOSURE

The present disclosure provides systems and methods capable of efficient and scalable production of platelets and other biological products.

In medical practice, various biological products can be used to treat various disorders, infections, malignancies, and traumas. Additionally, such biological products (e.g., plasma, platelets, white blood cells, red blood cells) can be used to replace depleted biological products within a patient. Production of such biological products has been attempted using various techniques such as production from various stem cells. Stem cells utilized have typically included embryonic stem cells, umbilical cord blood stem cells and induced pluripotent stem cells. Other stem cell sources have included stem cells found in bone marrow, fetal liver and peripheral blood. However, despite successful production of some biological products in the laboratory, many limitations remain to use in a clinical setting.

While substitute biological products (e.g., platelet substitute products or red blood cell substitute products) like lyophilized platelets (PLTs), cold-stored PLTs and infusible PLT membranes are under investigation, the considerable risks of bacterial contamination and immunogenicity posed by donor PLT-based products persist in these products, and they are still subject to short supply and limited storage life. Risks of febrile non-hemolytic reactions, alloimmunization-induced refractoriness, graft-versus-host disease, immunosuppression, and acute lung inflammation/injury can only partially be reduced by extensive screening and serological testing of donor blood and leukoreduction processes at considerable additional cost.

Synthetic biological products (e.g., synthetic PLTs or red blood cells) have been proposed as a solution and several designs have been studied which decorate synthetic particles with motifs that promote PLT-mimetic adhesion or aggregation. Recent refinement in these designs has involved combining the adhesion and aggregation functionalities on a single particle platform, and constructing particles that also mimic natural PLT's shape, size and elasticity, to influence margination and wall-interaction. The optimum design of a synthetic PLT analog would require efficient integration of platelet's physico-mechanical properties and biological functionalities. However, synthetic biological products pose 2 major complications.

First, synthetic biological products preferably mimic the biological properties of their mimicked biological products. For example, synthetic PLTs must specifically mimic the hemostatic properties and site-selective activation resulting in clotting and blood vessel healing over time. This is complicated by the observation that PLTs play multiple roles (both known and unknown) that include regulating inflammation, lymphatic and blood vessel repair, and tumor metastasis. Because synthetic PLTs focus on replicating the expression of single or paired receptors/proteins on the PLT surface, ‘activation’ is limited to very specific (and sometimes unpredictable) triggers, and produce only a partial physiological response. This has historically resulted in poor site-selectivity and a high risk of toxicity in subsequent clinical trials. By focusing on individual roles of PLTs in vivo, it is unlikely that synthetic PLTs will be able to completely reproduce the multiple functions (both known and unknown) that PLTs play in the body.

Second, the physico-mechanical properties of biological products, including their shape, size and mechanical modulus have been shown to significantly influence their functionality. For example, the shape, size and mechanical modulus of PLTs affect their circulation, distribution, cell-to-cell and cell-to-wall interactions under hemodynamic blood flow. Such properties are not easily reproduced artificially. While various designs for synthetic PLT substitutes have been proposed over the past 20 years they have consistently fallen short of the real thing.

By contrast, bioreactor-derived biological product production from a replenishable source of human biological source material (e.g., iPSCs for producing PLTs) addresses all of the limitations of existing biological product substitutes and synthetic biological products, and addresses the problems of biological product safety and unmet demand.

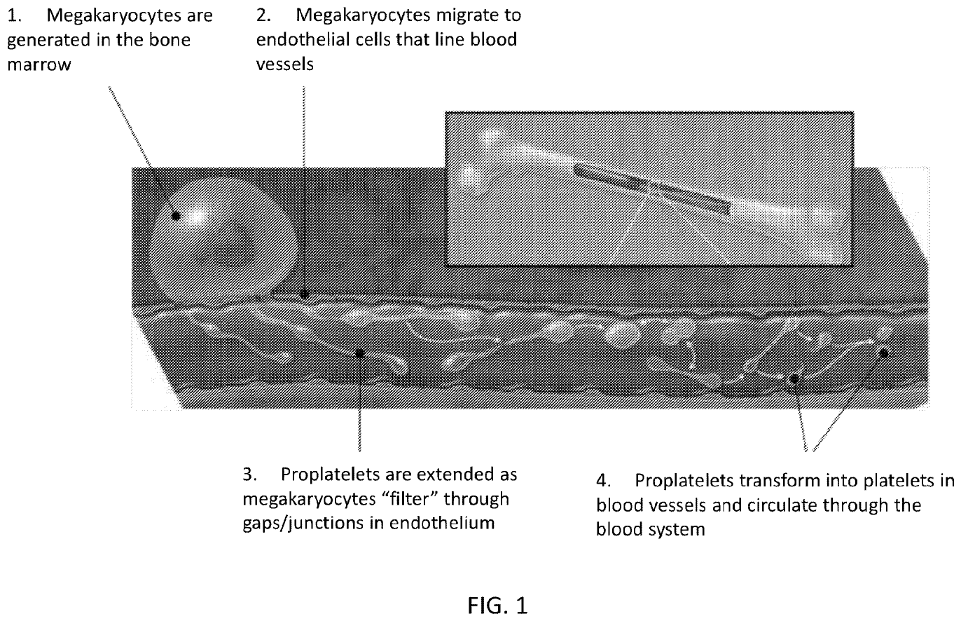

For example, blood platelets, or thrombocytes, are irregular, disc shaped cell fragments that circulate in the blood and are essential for hemostasis, angiogenesis, and innate immunity. In vivo, platelets are produced by cells, known as megakaryocytes. As illustrated in FIG. 1, megakaryocytes generated in the bone marrow move toward and settle onto endothelium cells that line blood vessels. There they extend long, branching cellular structures called proplatelets into the blood vessel space through gaps in the endothelium. Experiencing shear rates due to blood flow, proplatelets extend and release platelets into the circulation. In general, normal platelet counts range between 150,000 and 400,000 platelets per microliter of blood. However, when blood platelet numbers fall to low levels (e.g., below 150,000 platelets per microliter), a patient develops a condition known as thrombocytopenia and becomes at risk for death due to hemorrhage. Known causes for thrombocytopenia include malignancy and chemotherapy used to treat it, immune disorders, genetic disorders, infection, and trauma.

Despite serious clinical concerns for deleterious immune system response, risk due to sepsis and viral contamination, treatment of thrombocytopenia generally involves using replacement platelets derived entirely from human donors. However, the process of obtaining platelets from transfusions is lengthy, costly, and often requires finding multiple matching donors. In addition, the usability of harvested platelets are limited due to a short shelf-life on account of bacterial testing and deterioration. Moreover, we cannot currently screen for viruses we do not know exist. Combined with shortages created by increased demand and near-static pool of donors, it is becoming harder for health care professionals to provide adequate care for patients with thrombocytopenia, and other conditions related to low platelet counts. Alternatives to transfusion have included use of artificial platelet substitutes, although these have thus far failed to replace physiological platelet products (e.g., for the reasons explained above).

In some approaches, production of functional human platelets has been attempted using various cell culture techniques. Specifically, platelets have been produced in the laboratory using megakaryocytes obtained from various stem cells. However, despite successful production of functional platelets in the laboratory, many limitations remain to use in a clinical setting.

For instance, only approximately 10% of human megakaryocytes have been shown to initiate proplatelets production using state-of-the art culture methods. This has resulted in yields of 1 to 100 platelets per CD34+ cord blood-derived or embryonic stem cell-derived megakaryocyte, which are themselves of limited availability. For example, the average single human umbilical cord blood unit can produce roughly 5.106 CD34+ stem cells. This poses a significant bottleneck in ex vivo platelet production. In addition, cell cultures have been unable to recreate physiological microenvironments, providing limited individual control of extracellular matrix composition, bone marrow stiffness, endothelial cell contacts, and vascular shear rates. Moreover, cell cultures have been unsuccessful in synchronizing proplatelet production, resulting in non-uniform platelet release over a period of 6 to 8 days, which is on the order of platelet shelf-life. Furthermore, such inefficiencies result in high production costs due to combined costs associated with, for example, the required large quantities of fluid cell culture media, small molecules, cytokines, and growth factors. Methods of production of other biological products suffer from similar shortcomings.

Therefore, in light of the above, there remains a need for efficient ways to produce clinically relevant yields of biological products that can meet growing clinical demands, and avoid the risks and costs associated with donor harvesting and storage.

As will be apparent in view of this disclosure, a fluidic bioreactor, for example, a millifluidic bioreactor or a microfluidic bioreactor, can be used to support cell culture. It will be understood that any flow rate through a bioreactor can be used to achieve cell culture depending on the cell type and desired yield. In accordance with various embodiments, the bioreactor can support high yield cell culture at much smaller volumes, which enables substantial cost reductions in cell culture. Such cost reductions can provide commercially feasible, cost efficient production of biological products, thereby permitting translation of production processes to commercially feasible industrial production for clinical use.

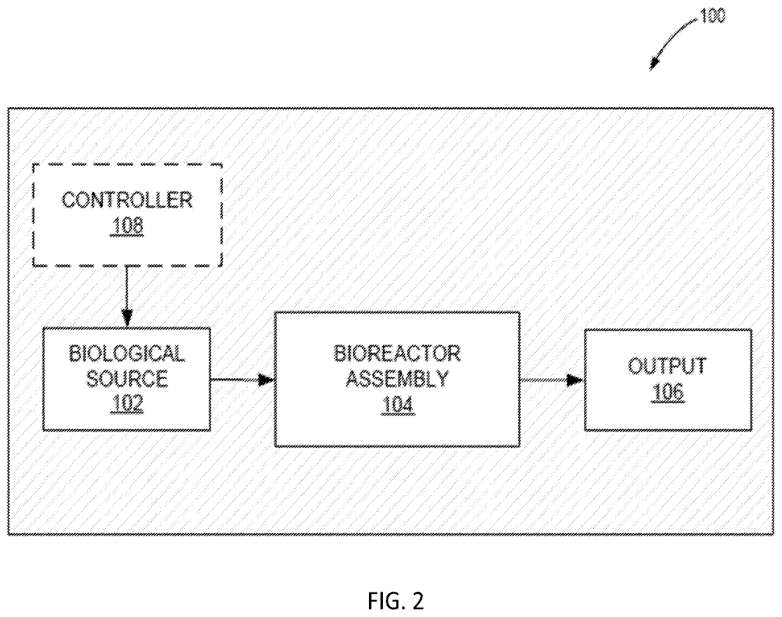

Turning now to FIG. 2, a schematic diagram of an example system 100 for producing platelets, and other biological products, is shown. In general, the system 100 includes a biological source 102, a bioreactor assembly 104, and an output 106, where the biological source 102 and output 106 are connectable to various inputs and outputs of the assembly 104, respectively.

Specifically, the biological source 102 can be configured with various capabilities for introducing into the assembly 104 different biological source materials, substances, gas, or fluid media, to efficiently produce desirable biological products, such as, for example, platelets. For instance, the biological source 102 can include one or more pumps for delivering, sustaining, and/or recirculating fluid media in the bioreactor assembly 104. Examples include but are not limited to fluidic pumps, syringe pumps, peristaltic pumps, pneumatic pumps, and the like. The biological source materials can include but are not limited to cells, cell culture media, small molecule compounds, gases and gas mixtures, and nutrients.

As shown in FIG. 2, in some embodiments, the system 100 can also include a controller 108 for controlling the biological source 102. Specifically, the controller 108 can be a programmable device or system configured to control the operation of the bioreactor assembly 104, including the timings, amounts, and types of biological source material, substances, fluid media or gas introduced therein. In some aspects, the controller 108 can be configured to selectively functionalize and/or operate the assembly 104 to recreate physiological conditions and processes associated with cell differentiation (e.g., platelet production) in the human body. For example, the controller 108 can be programmed to deliver a selected number of megakaryocytes to the assembly 104. In addition, the controller 108 can control fluid flow rates or fluid pressures in the bioreactor assembly 104 to facilitate proplatelet extension and platelet production. For instance, the controller 108 can establish flow rates up to 150,000 microliters/hr in various channels configured in the bioreactor assembly 104, or any flow rate necessary to establish a local shear rate that triggers platelet production from the seeded megakaryocytes, or other biological products.

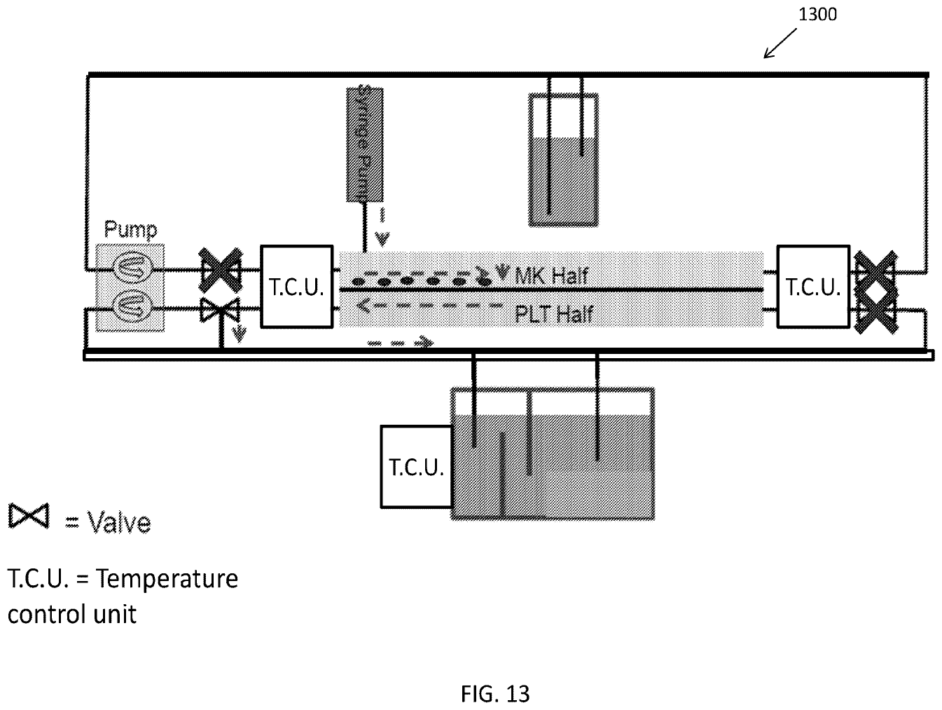

Although the controller 108 is shown in FIG. 2 as separate from the biological source 102, it can be appreciated that these can be combined into a single unit. In some embodiment, the biological source 102 and controller 106 can be embodied in a programmable fluidic pump or injection system. In addition, in some implementations, the controller 108 and/or biological source 102 can also include, communicate with, or received feedback from systems or hardware (not shown in FIG. 2) that can regulate the temperature, light exposure, vibration, pressure, shear rate, shear stress, stretch, and other conditions of the bioreactor assembly 104. By way of example, FIG. 13 shows a bioreactor system 1300 that includes a temperature control units (T.C.U) in communication with one or more heaters and one or more thermocouples for maintain, monitoring and controlling temperature. It can be appreciated that the configuration shown in FIG. 13 is non-limiting, and any number of heaters, and heater arrangements can be possible. It will be further apparent that, in accordance with various embodiments, any number of additional components can also be included, such as, for example, pressure sensors, in-line pressure readers, or any other suitable component.

Referring again to FIG. 2, in general, the output 106 is configured to receive fluid media containing various biological products generated in the bioreactor assembly 104. In some embodiments, such effluent can be redirected or circulated back into the bioreactor assembly 104. In this manner, less fluid volume may be utilized, and the biological products generated can be more concentrated. In some aspects, the output 106 can also include capabilities for collecting, storing and/or further processing received fluid media. In some embodiments, such features can advantageously improve efficiency of the biological product generation process, thereby reducing manufacturing costs.

It can be appreciated that the above-described system 100 has a broad range of functionality, and need not be limited to replicating physiological conditions or processes, nor producing platelets. That is, the system 100 can be used to generate a wide variety of biological products. For instance, the system 100 can be used to support cell culture and/or separate various biological source materials or substances, cells at various stages of their differentiation process, and collect their products or content. Specifically, by controlling media composition, fluid flow, and pressures, as well as other conditions, various biological source materials may be produced and released and subsequently harvested. Example biological products include but are not limited to isolation of cells from cell mixture or at various stages of differentiation, growth factors, antibodies, and other components found in cells. Controlling operating conditions such as temperature, pH, concentration of compounds or proteins, can be used to influence the properties of the biological products, such as platelet activation state, platelet compound or protein loading, protein conformation, and product yield. Produced biological products, in accordance with the present disclosure, in addition to clinical use, can find use in a variety of applications including isolation of cells from cell mixture, differentiation of cell progenitors, generation of tissues, and as components of cell culture medias and cosmeceuticals, such as cosmetics, shampoos, skin additives, creams, or cleaners, and so forth.

Various embodiments of the above system 100 will now be described. It can be appreciated that these are non-limiting examples, and indeed various modifications or combinations are possible and considered by one of ordinary skill in the art to be within the intended scope of the present application.

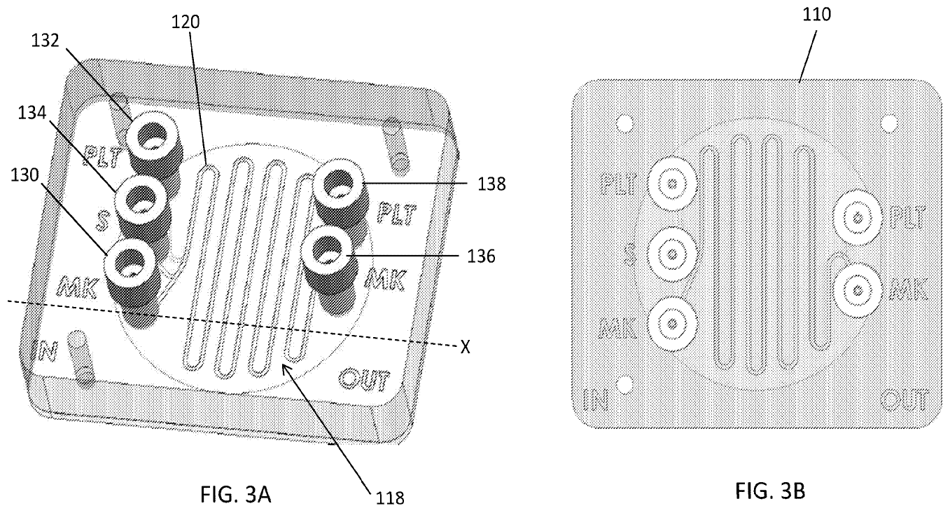

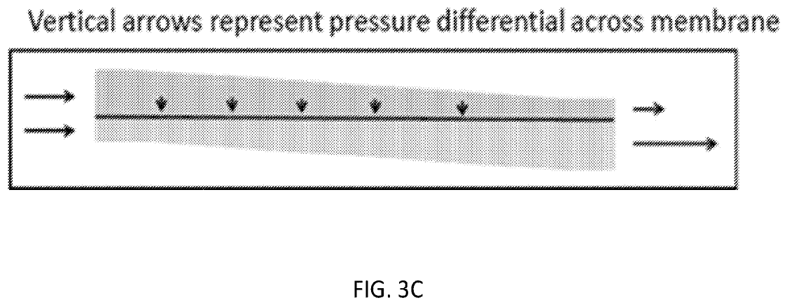

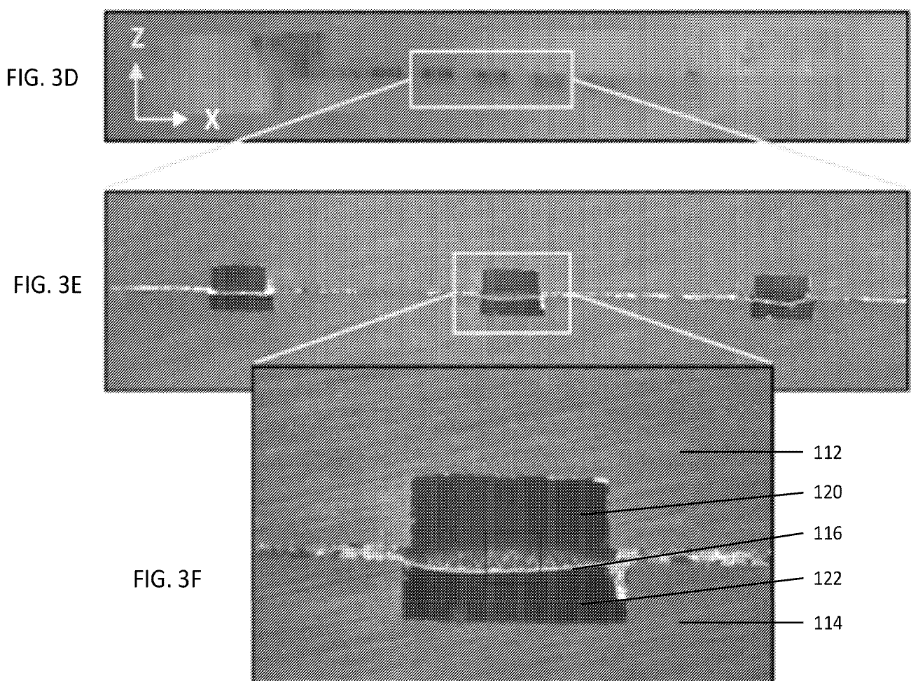

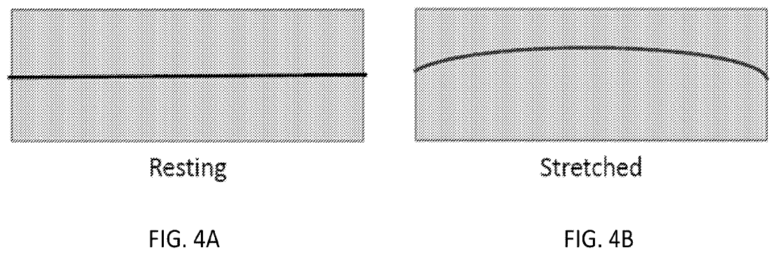

Referring now to FIGS. 3A-3F, a bioreactor is shown in accordance with various embodiments. In some embodiments, the bioreactor 104 includes a bioreactor body 110 including a first substrate 112, an opposing second substrate 114 engaged with the first substrate 112, and a membrane 116 arranged at least partially therebetween. As shown, for example, in FIG. 3F, the membrane may be made of a flexible material, or a material that can allow the membrane to stretch or curve under pressure. In some embodiments, this may further assist in adjusting the pressure through the membrane, while avoiding damaging the biological source material that can be distributed on the surface of the membrane or inside the membrane. In some embodiments, the bioreactor body 110 can include a serpentine pathway 118 extending therethrough and formed by a first channel 120 defined in the first substrate 112 and a second channel 122, aligned with the first channel 120, defined in the second substrate 114.

In some embodiments, the bioreactor 104 can further include a plurality of inlets (at least one inlet per channel) and a plurality of outlets (at least one outlet per channel). For example, as shown in FIGS. 3A-3B, in some embodiments the bioreactor 104 can include a first inlet 130 for providing a first fluid flow to the first channel 120, a second inlet 132 for providing a second fluid flow to the second channel 122, and a third inlet 134 for introducing a biological source material into the first channel 120. As further shown in FIGS. 3A-3B, in some embodiments the bioreactor 104 can include a first outlet 136 for permitting the first flow to exit the first channel 120 and a second outlet 138 for permitting the second flow to exit the second channel 122. It will be apparent, however, that any number of inlets and outlets can be provided for supplying or removing fluids or materials into and out of the channels and that additional conduits can also be formed in the substrates of the bioreactor of FIGS. 3A-3F. For instance, in some embodiments, a perfusion channel can also be included in the bioreactor. In such embodiments, the perfusion channel can, for example, allow for the flow of a gas, which can subsequently perfuse through the substrate materials and into the first and second bioreactor channels. For example, in some embodiments, a gas mixture having about 5% CO₂ to about 10% CO₂ can be perfused into one or more of the channels to provide appropriate pH buffering. In some embodiments, a gas mixture having about 4% O₂ to about 20% O₂ can be perfused into one or more of the channels to provide appropriate oxygen content for cells with different metabolic needs. In some embodiments, a gas mixture including about 4% O₂ and about 10% CO₂ can be perfused into one or more of the channels and can be used for various cell differentiation applications. In some embodiments, a gas mixture including about 20% O₂ and about 5% CO₂ can be perfused into one or more of the channels and can be used for various cell growth applications.

The input materials that enter the inlets of the upper chamber can include cells and cell mixtures, cell culture media, buffer, protein solutions, and small molecule compounds. The components of such inputs which size is above the size of the membrane of the bioreactor will remain in the upper chamber, while those below that threshold will be allowed to pass to the lower chamber. The inputs of the lower chamber can include cell culture media, buffer, protein solutions, and small molecule compounds. The output of the upper chamber, which can operate open or closed, can include products of the input materials such as platelets or proteins, as well as the input materials such as cells and culture media. The output of the lower chamber can include products of the inputs from the upper chamber that are below the size of the membrane pores, as well as products from the inputs into the second chamber. For example, platelets and proteins. For examples, in FIG. 3, the upper channel inlets are the MK and S inlet, the upper channel outlet is the MK outlet, the lower channel inlet is the PLT inlet, and the lower channel outlet is the PLT outlet.

The substrates, in some embodiments, can be of any suitable size, including, for example, having lateral dimensions in a range between 10 mm and 100 mm, and a thickness in a range between 1 and 10 mm, although other dimensions can be used in accordance with various embodiments. The substrates, in some embodiments can be manufactured using any combination of biocompatible materials, inert materials, as well as materials that can support pressurized gas and fluid flow, or gas diffusion, and provide structural support. In some aspects, materials utilized in the bioreactor can be compatible with specific manufacturing processes, such as insert casting. In addition, materials utilized can optically clear to allow visualization of fluid media, and other substances, present or flowing in various portions of the bioreactor. The substrates, in some embodiments, can be constructed of, for example, one or more of polymethyl methacrylate (PMMA), polydimethylsiloxane (PDMS), one or more polycarbonates (PC), cyclic olefin copolymer (COC), cyclic olefin polymer (COP), polyvinyl chloride (PVC), coated polystyrene, coated glass, polyurethane (PU), silicone elastomers, or combinations thereof. The substrates and channels, in accordance with various embodiments, can be constructed by one or more of machining flow channels into block base material, casting, hot embossing, soft lithography, thermoforming, insert casting, or combinations thereof. The substrates can be assembled with the membrane therebetween, for example, by application of two pieces of laser cut pressure sensitive adhesive to adhere the substrates to each side of the membrane, mechanical clamping, solvent bonding, thermal bonding, diffusion bonding, combinations thereof, or even manufactured in one piece through injection molding.

The channels formed through the substrates can vary in length, size, and shape. In some embodiments, a length of each of the first and second channels can be in the range of about 10,000 to about 1,000,000 micrometers or, more particularly, in the range of about 25,000 to about 320,000 micrometers, while at least one transverse dimension of the each of the first and second channels can be in the range of about 100 to about 3,000 micrometers or, more particularly, in the range of about 500 to about 1,000 micrometers. However, it will be understood that first and second channels can be of any length or width in accordance with various embodiments. In some embodiments, the first and second channels can have identical length and transverse dimensions. In some embodiments, the first and second channels can have different widths and/or transverse dimensions. For example, in some embodiments, the first channel can be wider or narrower than the second channel. These geometrical modifications can be used to control the fluid dynamic properties of each channel independently. Furthermore, in some embodiments, as shown in FIG. 3D which illustrates a cross-sectional view of the bioreactor of FIG. 3A through line X, each channel can also be tapered either over the entire length of the serpentine pathway or over a portion of the length of the serpentine pathway to control shear rates or pressure differentials between the channels, over an active contact area, regulating perfusion through the membrane.

Various implementations of the bioreactor are possible depending upon specific uses or applications. For example, dimensions, shapes, and other features of various components of the bioreactor can be selected based on the desired output of the bioreactor. In particular, in some embodiments, the first and second channels, along with other fluidic elements of bioreactor, can be shaped and dimensioned to reproduce physiological conditions, such as those found in bone marrow and blood vessels. In some embodiments, channel shapes and dimensions can be selected to achieve physiological flow rates, shear rates, fluid pressures and/or pressure differentials similar to those associated with, for example, in vivo platelet production, as described with reference to FIG. 1.

In reference to FIG. 3C, in some embodiments, the bioreactor can operate by having a pressure drop between the inlets and outlets that drives fluid from the inlets to the outlets. The geometry of the one or both of the channels can be adjusted to set the pressure differential across the membrane to be uniform throughout the entire channel. The geometry of the one or both channels can also be adjusted to achieve constant or near constant shear rates at the membrane through the entire length of the device channels. For example, in a regime where the width (w) of the channel is much larger than its height (h), the shear rate at the membrane (τ) can be held approximately constant across the length of the channel, where the volumetric flow rate (Q) is decreasing or increasing, by decreasing or increasing the height of the channel following the relationship:

$$τ = \frac{6 Q}{{wh}^{2}}$$

In some embodiments, the instant bioreactor can be configured such that the pressure and shear stress are decoupled and can vary independently of one another. In some embodiments, the instant bioreactor is designed such that the shear stress can be altered by changing the operating flow rate, while the transmembrane pressure associated with such flow rate change can be offset by decreasing the flow through the membrane, by, for example, modifying the number of occluding the membrane pores. Alternatively, the shear rate can be increased by increasing flow rate, while pressure across the membrane can be kept constant by decreasing the cell seeding density. This can enable identification of appropriate regimes of biophysical parameters that allow for specific biological processes, such as platelet production.

In some embodiments, the first channel, the second channel, or both can be sized and shaped to ensure uniform seeding of the biological source material over the membrane. In some embodiments, such uniform seeding can be achieved by maintaining the near constant shear distribution along the membrane by the decreasing height of the channel, as well as maintaining a constant pressure differential across the membrane. This can be achieved on either side of the membrane by inverting the direction of the flow on both channels.

In addition, configurations of the bioreactor can be chosen to allow cooperation with other instrumentation, such as microscopes or cameras. For instance, the bioreactor can be configured to adhere to standard microplate dimensions. However, it will be apparent in view of this disclosure that any number of dimensions or configurations can be used in accordance with various embodiments to permit connection to any number and type of instruments, operational infrastructure devices, and/or additional bioreactors.

In some embodiments, the first and second channels terminate in their respective substrates to create a single fluid conduit from the first inlet to the first outlet and from the second inlet to the second outlet, respectively, as shown in FIGS. 3A-3F. That is to say, fluid media introduced into the first inlet is necessarily extracted from the first outlet and fluid media introduced into the second inlet is necessarily extracted from the second outlet. However, it will be appreciated in view of this disclosure that additional inlets and outlets can also be possible with the bioreactor and connected to the first and second channels or any additional channels through the substrate (e.g., the third inlet as shown in FIGS. 3A-3F). Additional inlets can be used to introduce different biological materials. Additional outlets can be used to, for example, fractionate the outcome of biological products, either uniformly or making use of differences in intrinsic properties of the products affecting their positioning in the channels.

The membrane formed between the first channel and the second channel can be formed in a variety of ways. In some embodiments, the membrane can include any rigid or flexible layer, film, mesh or material structure configured to connect corresponding inlet channel and outlet channel via fluidic pathways formed therein. In some embodiments, the membrane can be formed from any suitable material including, for example, Polycarbonate Track Etch—Polyvinylpyrrolidone Free (PCTEF), Polycarbonate Track Etch—PVP Coated (PCTE), hydrophilic polycarbonates, hydrophobic polycarbonates, polyvinyl chloride (PVC), polyester, cellulose acetate, polypropylene, PTFE, polyurethane (PU), silicone elastomers, or combinations thereof. In some embodiments, fluidic pathways in the membrane can be formed using pores, gaps or microchannels, distributed with any density, either periodically or aperiodically, about membrane. In some embodiments, the membrane can include a three-dimensional structure formed using interwoven micro- or nano-fibers arranged to allow fluid therethrough. Although shown in FIGS. 3A-3F as rectangular in shape, it can be appreciated that the membrane can have any shape, including circular shapes, oval shapes, and so forth. In accordance with aspects of the disclosure, the membrane can be configured to selectively capture specific biological source materials or substances to produce desired biological products. For instance, the membrane may be configured to selectively capture platelet-producing cells and allow proplatelet extensions and platelets therethrough.

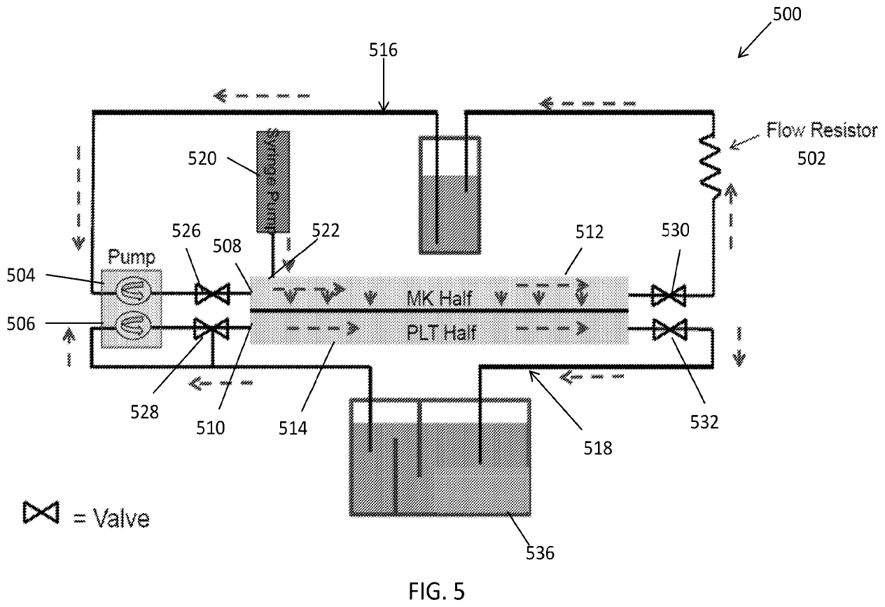

In some embodiments, the membrane can be flexible to more closely mimic pulsatile blood flow within a patient. In such embodiments, as shown in FIGS. 4A-4B, the membrane can transition between a substantially planar resting position during a resting pulse, as shown in FIG. 4A, and a stretched configuration during a pressure pulse, as shown in FIG. 4B.

The dimensions of the membrane are also variable. In some embodiments, the membrane can include longitudinal and transverse dimensions in a range between about 1 and about 100 millimeters, and have a thickness in a range between about 0.1 to about 20 micrometers, although other dimensions are possible. Also, the membrane can include pores, gaps or microchannels sized in a range between about 1 micrometers and about 20 micrometers, for example, about 5 to about 8 micrometers. In some embodiments, pore, gap or microchannel size, number, and density can depend on a number of factors, including but not limited to desired biological products and product yields, as well as flow impedances, shear rates, pressure differentials, fluid flow rates, and other operational parameters. In some embodiments, the membrane can include pores, gaps, or microchannels in a density of about 500 to about 10,000 pores per mm².

As will be appreciated from FIGS. 3A-3F, the opposing first and second channels are in overlapping alignment to define an active contact area in the membrane. For example, an active contact area may be in a range between about 1 mm² to about 20 mm², although other active areas are possible, depending upon the dimensions and number of channels utilized. In some implementations, the active contact area along with membrane characteristics can be optimized to obtain a desired biological product yield. For example, a membrane with 47 mm diameter, 5% active contact area, and pore density of about 1·10⁵ pores/cm² could provide about 200,000 potential sites for generating a desired biological product yield, such as a desired platelet yield. In some applications, the active contact area can be configured to trap at least about 1·10⁴ megakaryocytes.

The substrates and membrane can be manufacture by a number of different processes. By way of example, the first substrate, or second substrate, or both, or portions thereof, can be manufactured using cell-inert silicon-based organic polymer materials, such as polydimethylsiloxane (“PDMS”), thermoplastic materials, such as cyclo olefin polymer (“COP”), glass, acrylics, and so forth. On the other hand, the membrane 116 can be manufactured using PDMS, thermoplastics, silk, hydrogels, extracellular matrix proteins, polycarbonate materials, polyesthersulfone materials, polyvinyl chloride materials, polyethyleneterephthalat materials, polyurethane (PU), silicone elastomers, and other synthetic or organic materials. Additionally, the bioreactor can be manufactured as one piece and as a series of bioreactors, through processes such as injection molding.

In some embodiments, the bioreactor can be functionalized to replicate in vivo physiological conditions in order to produce biological products such as, for example, platelets. For example, various substances can introduced onto a surface of the membrane or into one or more of the channels to affect the reactions within the bioreactor. In some embodiments, a top surface of the membrane can be selectively coated with extracellular matrix proteins or functional peptides or other molecules, for example, while a bottom surface can be left without, or can be coated with different proteins or substances. In some embodiments, one or both channels can be filled with a hydrogel trapping cells and other materials in a 3D matrix. Selective perfusion of media in one channel wherein the second channel contains a hydrogel can, for example, create a concentration gradient in the gel that can be used to direct cell migration or differentiation or study small molecule, cytokine, growth factor diffusion.

Such coatings can be achieved, for instance, by infusing a first fluid medium containing extracellular matrix proteins, using inputs and outputs in the first substrate. At substantially the same time, a second fluid medium flow can be maintained in the second substrate using respective inputs and outputs, where the second fluid medium can either contain no proteins, or different proteins or substances. In some embodiments, flow rates of the first and second fluid media can be configured such that little to no fluid mixing would occur. Such selective functionalization can ensure that introduced platelet-producing cells, for example, coming to rest on the top surface can contact extracellular matrix proteins, while proplatelets extend through the membrane, and platelets released therefrom, would not contact extracellular matrix proteins, or would contact different proteins or biological substances. In some embodiments, the membrane can instead be pre-coated before assembly within the bioreactor.

Non-limiting examples of biological substances and materials for functionalizing the bioreactor can include human and non-human cells, such as megakaryocytes, endothelial cells, bone marrow cells, osteoblasts, fibroblasts, stem cells, blood cells, mesenchymal cells, lung cells and cells comprising basement membranes. Other examples can include small molecules, such as CCL5, CXCL12, CXCL10, SDF-1, FGF-4, VEGF, Flt-3, IL6, 9, 3, 1b, TPO, S1PR1, RGDS, Methylcellulose. Yet other examples can include, extracellular matrix proteins, such as bovine serum albumin, collagen type I, collagen type IV, fibronectin, fibrinogen, laminin, vitronectin (PLL), or any peptide sequences derived from these molecules. In particular, to replicate three-dimensional extracellular matrix organization and physiological bone marrow stiffness, cells can be infused in a hydrogel solution, which may subsequently be polymerized. The hydrogel solution may include, but is not limited to alginate, matrigel, agarose, collagen gel, fibrin/fibrinogen gel, and synthetic gels such as polyethylene glycol gels.

In some embodiments, various portions of the bioreactor can be configured to allow for assembly and disassembly. In some embodiments, the first substrate, membrane, and second substrate can be configured to be removably coupled to one another. When engaged using fasteners, clips, or other releasable locking mechanisms, for example, a hermetic seal can then be formed between various surfaces of the substrates and membrane to reinstate fluid pathway integrity between the first and second inlets and the first and second outlets. It will be understood that any type of connector can be used to connect the various components of the bioreactor as long as a hermetic seal can be achieved. This capability can facilitate preparation, as described above, as well as cleaning for repeated use. In addition, disassembly allows for quick exchange of various components, for repurposing or rapid prototyping. For instance, a membrane having different pore sizes, or different preparations, can be readily swapped.

Alternatively, the bioreactor can be manufactured as a unitary device. In some embodiments, the bioreactor can be formed as a unitary device using an insert casting technique or an injection molding technique, where the membrane can be molded into the substrates. Such implementations can be advantageously integrated into large scale manufacturing techniques. In some embodiments, the first and second substrates can be manufactured separately and them bonded together by using an adhesive and/or thermal bond to permanently couple the first substrate, the membrane, and the second substrate together. It will be understood that any technique can be used to produce a unitary bioreactor, which may be desirable to ensure that there is no leakage from the channels of the bioreactor.

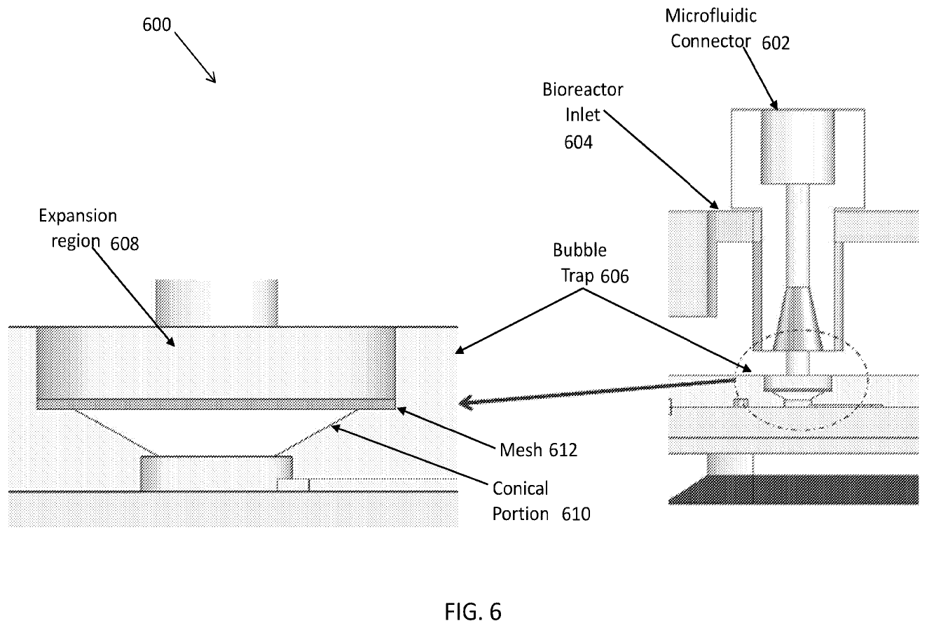

The bioreactor can also include a number of fluidic filtration and resistive elements, connected to the channels and arranged at various points along the various fluid pathways extending between the inlets and outlets. FIG. 5 illustrates an exemplary embodiment of a bioreactor 500 that includes filtration and resistive features. For instance, one or more filtration elements (not shown) can be placed proximate to one or more of the inlets to capture contaminants or undesirable substances or materials from an inputted fluid medium. In addition, one or more resistive elements 502 can also be included to control flow forces or damp fluctuations in flow rates. In addition to resistive and filtration elements, additional elements can also be included. For example, one or more of the inlets can include bubble traps configured to prevent any air bubbles from entering the bioreactor. In some embodiments, one or more of the inlets can include an in-line mixer for, for example, homogenizing the first fluid flow with the biological source material or, for example, homogenizing the second fluid flow with the biological products.

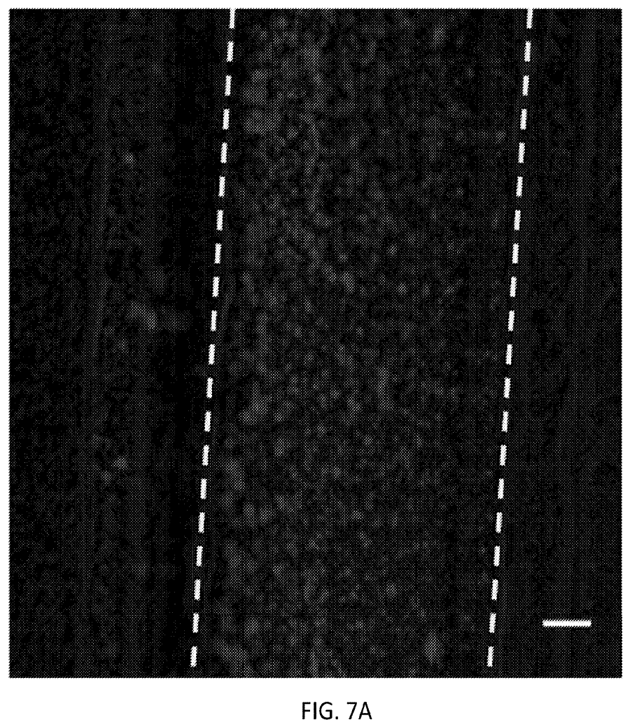

By way of a non-limiting example, FIG. 6 illustrates a port 600 that includes fluidic connector or port 602 coupled to an inlet 604 of an exemplary bioreactor, in accordance with various embodiments. As shown, the inlet 604 has a bubble trap 606 that includes an expansion region 608 and a conical region 610 separated by a mesh 612. The size of the mesh 612 can vary, but in some embodiment the mesh 612 can have a size of approximately 140 micrometers, although other values can be possible. As configured, the bubble trap is capable of preventing air bubbles from entering the bioreactor.

As shown in FIG. 5, in some embodiments, a bioreactor can be included in a recirculating bioreactor 500. In some embodiments, the recirculating bioreactor 500 can include a bioreactor 104 as described above with reference to FIGS. 3A-3F. In some embodiments the recirculating bioreactor 500 can include first and second pumps 504, 506 for recirculating flow from the first and second outlets 512, 514 back to the first and second inlets 508, 510 via first and second recirculation lines 516, 518. In some embodiments, the recirculating bioreactor can include a third pump 520 (e.g., a syringe pump as shown) for delivering a biological source material to the first channel 524 via the third inlet 522. In some embodiments, one or more valves can be positioned in fluid communication with each of the inlets and outlets to permit, prevent, or control flow thereto. In the illustrated embodiment, each inlet 508, 510 and each outlet 512, 514 are associated with a valve 526, 528, 530, 532. In some embodiments, one or more reservoirs 532, 536 can be included to store excess fluid media of the first and/or second flows during operation. In some embodiments, at least one reservoir can be configured to separate a biological product from the second flow. In some embodiments, one or more flow resistors 502 can be added to one or more of the recirculation lines to provide additional control over flow rates and pressures within the bioreactor.

The first and second pumps 504, 506, in accordance with various embodiments, can be any suitable pump capable of imparting motive energy to the first and second fluid flows to promote flow through the first and second channels and first and second recirculation lines 516, 518. For example, in some embodiments the first and second pumps 504, 506 can include one or more of an impeller, a peristaltic pump, positive displacement pump, gear pump, screw pump, any other suitable pump, or combinations thereof. In some embodiments, each of the first and second pumps 504, 506 can be separately operable and reversible in order to provide independent flow control in each of the first and second channels. In some embodiments, the pumps 504, 506 can be configured to vary one or more of pressure, flow, and/or shear within each of the first and second channels to provide pulsatile flow through the bioreactor. In some embodiments, the pulse rate, pressure, shear, and/or flow can be provided to substantially mimic human blood flow. For example, in some embodiments, during operation, the perfusion rate of the flow circulating within the first and second channels can be between about 1 mL/hr and about 50 mL/hr, for example, about 12.5 mL/hr and produce a wall shear rate between about 250 s⁻¹ and about 1800 s⁻¹, for example 800 s⁻¹ and about 1200 s⁻¹. Pulse rate, in accordance with various embodiments, can be about 0.5 hz to about 5 hz, for example, about 1 hz to about 2 hz.

The third pump 520, in accordance with various embodiments, can be any pump suitable or infusing a biological source material into the first channel. For example, in some embodiments the third pump 520 can be a syringe pump, a piston pump, a reciprocating pump, a diaphragm pump, any other suitable pump, or combinations thereof. In some embodiments, the third pump 520 can be configured to deliver the biological source material at a rate sufficient for seeding the membrane with the biological source material. For example, in some embodiments the biological source material can be infused at a rate of about 0.1 mL/hr to about 2 mL/hr, for example, about 1 mL/hr. However, it will be apparent in view of this disclosure that any suitable flow rate can be used in accordance with various embodiments.

The flow resistor 502, in accordance with various embodiments can include, for example, a nozzle, a tube extension, any other device suitable for metering or restricting fluid flow, or combinations thereof. The valves, in some embodiments, can be any valve known in the art for selectively permitting or preventing flow through the first or second channels and/or the first or second recirculation lines. The first and second reservoirs can be any suitable beaker, test tube, flask, bottle, jar, tank, or any other suitable reservoir for retaining a fluid medium. In some embodiments, the second reservoir can further include at least one of a divider, a separator, a sorter, or any other device for removing one or more biological products from the second fluid flow, such as a hollow fiber or cross filtration device.

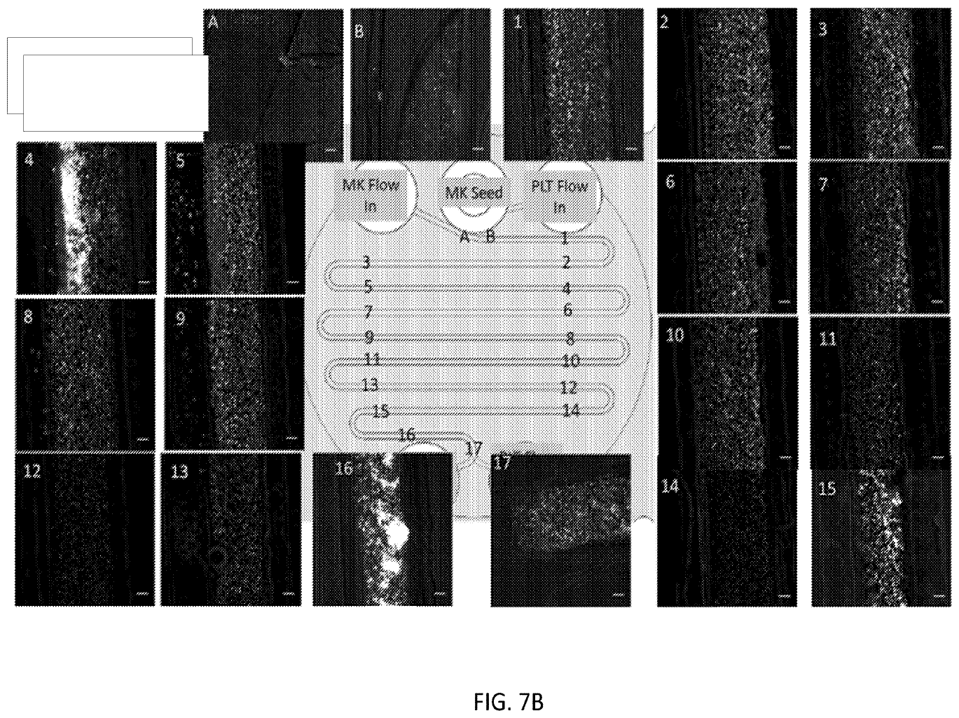

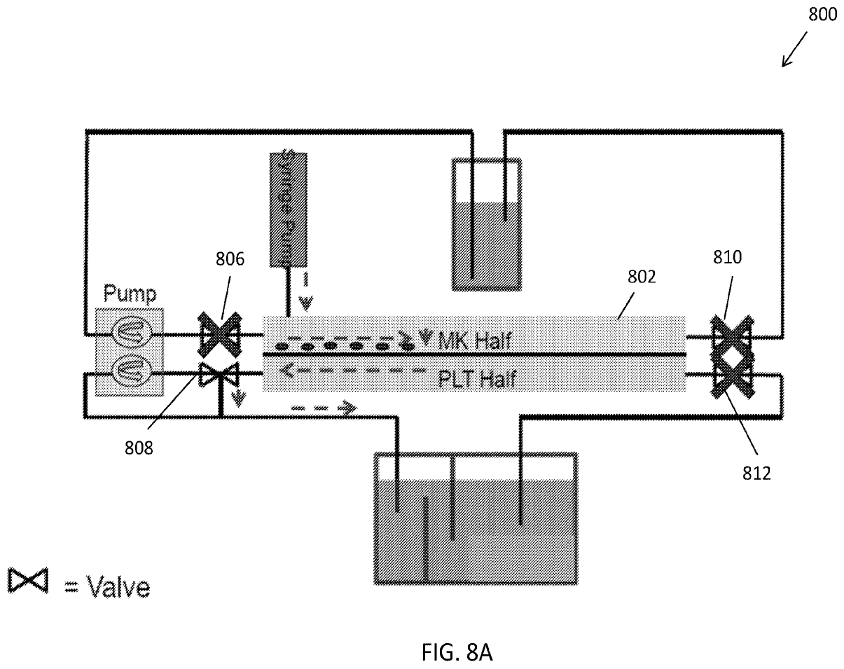

Recirculating bioreactors as described above with reference to FIGS. 3A-3F, 4A-4B, and 5 can provide a uniform seeding of the membrane with biological source material along the length of the first channel. For example, FIG. 7A is an exemplary image showing megakaryocyte distribution along a section of a bioreactor channel in accordance with various embodiments. As shown, rather than clustering in one specific localized area, the megakaryocytes are substantially uniformly distributed across and along the membrane. FIG. 7B illustrates exemplary megakaryocyte distribution at various stations along a bioreactor channel in accordance with various embodiments. As shown in FIG. 7B, rather than clustering in one specific localized area, the megakaryocytes are substantially uniformly distributed across and along the membrane at each station but also substantially uniformly distributed between each of the stations along the length of the bioreactor channel. Such uniform distributions of seeded biological source material can be achieved in a plurality of ways, including, for example, one or more of the methodologies described below with reference to FIGS. 5, 8A-8B, and 9.

By way of a non-limiting example, as shown in FIG. 5, a bioreactor of the present disclosure can be seeded using a double flow seeding technique. In the double flow seeding technique biological source materials dispersed in a fluid media are added to the first channel via the third inlet and with both pumps in operation such that the first fluid flow is provided through the first channel and the first recirculation line and the second flow is provided through the second channel and the second recirculation line. In order to prevent or reduce continuous recirculation (without capture by the membrane) of the biological source material, the flow resistor can be activated to increase pressure across the membrane. The double flow seeding technique can advantageously provide a more even distribution of the biological source material compared to the direct infusion methodology.

Furthermore, the operational configuration depicted in FIG. 5 can also be used after seeding, regardless of the seeding technique used, for actual operation of the recirculating bioreactor to produce biological products. In some embodiments, a flow resistor 502 can be used, for example, to increase pressure in the first channel by increasing the pressure drop between the first channel and the outlet. In some embodiments, the flow resistor 502 can be provided as a length of tubing having an inner diameter large enough for a seeding cell to pass through but small enough (and long enough) to create a desired rise in pressure. The increased pressure in the first channel can create a pressure differential for holding the seeded biological source material against the membrane pores, thereby permitting the seeded biological source material to maintain their position in a membrane pore and not be swept away or dislodged by other forces such as higher operational fluid media flow rates.

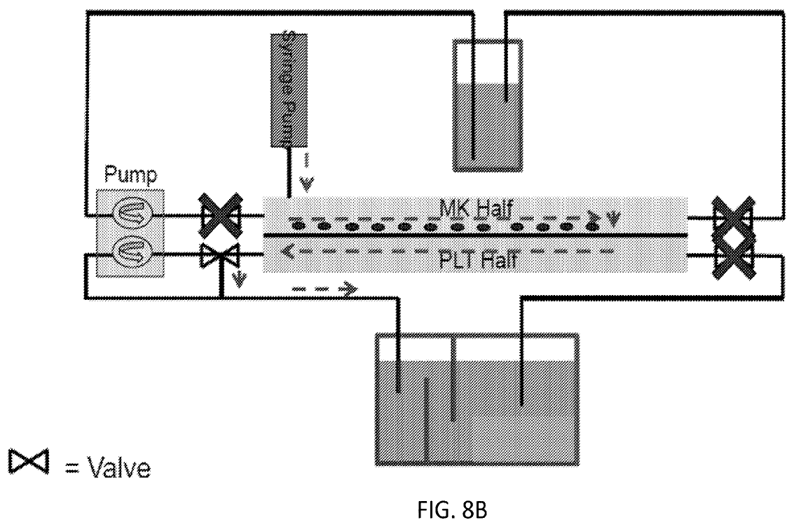

FIGS. 8A and 8B illustrate an embodiment of a recirculating bioreactor 800 seeded using a pressure wave seeding technique. In the pressure wave seeding technique, biological source materials dispersed in a fluid media are added to a first channel 802 via the third inlet with valves 806, 810, 812 controlling the first inlet, the first outlet, and the second outlet closed and the valve 808 controlling the second inlet open. Although described herein as closed, the valve 806 associated with the first inlet, in some embodiments, can be minimally open. For example, in some embodiments, one or both of the first or second inlets can be about 10% open to permit a small flow therethrough, thus preventing inadvertent collection of biological source material in the first or second inlets. The fluid media is then flowed from the third inlet to the second inlet. Because of the valve closures, the first inlet and first and second outlets are blocked. Thus, fluid media passes through the membrane to exit the bioreactor 800. Because the pores in the membrane are sized and configured to capture the biological source material, the biological source material becomes lodged in the pores of the membrane. Initially, as shown in FIG. 8A, the biological source material is captured by the closest pores to the third inlet and then, as the closest pores are blocked, subsequent biological source material travels through the channel to reach the next available open pores as shown in FIG. 8B. Accordingly, the pressure wave seeding method lays down a layer of cells with one cell on each pore as flow is gradually blocked through the membrane. The pressure wave seeding method advantageously provides even placement of the biological source material throughout the membrane. Additionally, because the flow is gradually blocked through the membrane, by measuring the pressure drop across the membrane during this process, the number of open and filled pores can be estimated.

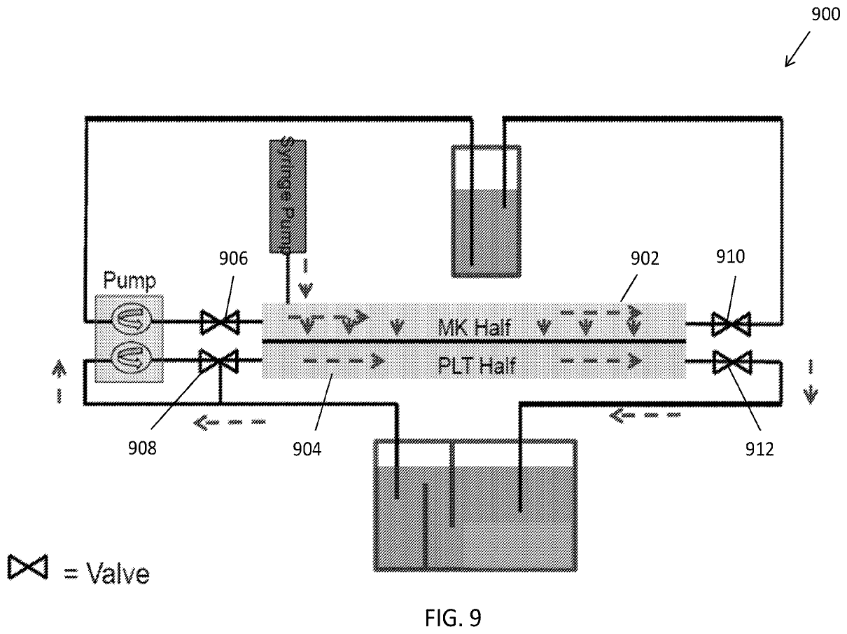

FIG. 9 illustrates an embodiment of a recirculating bioreactor 900 seeded using a direct infusion seeding technique. In the direct infusion seeding technique biological source materials dispersed in a fluid media are added to the first channel 902 via the third inlet with all valves 906, 908, 910, 912 open. The first pump 914 is inactive and the second pump 916 is operated to provide flow through the second channel 904 and the second recirculation line. This method prevents the biological source material from being recirculated during seeding because the first pump 914 is inactive. In some embodiments, direct infusion seeding results in concentrations of biological source material proximate the first inlet and the first outlet, with relatively little biological source material in the middle portions of the bioreactor. It will be apparent in view of this disclosure, however, that in some embodiments, the first pump, to prevent inadvertent collection of biological source material at the first inlet, can be operated at a speed slow enough to avoid recirculation of the biological source material, for example, about 10% operational flow rate.

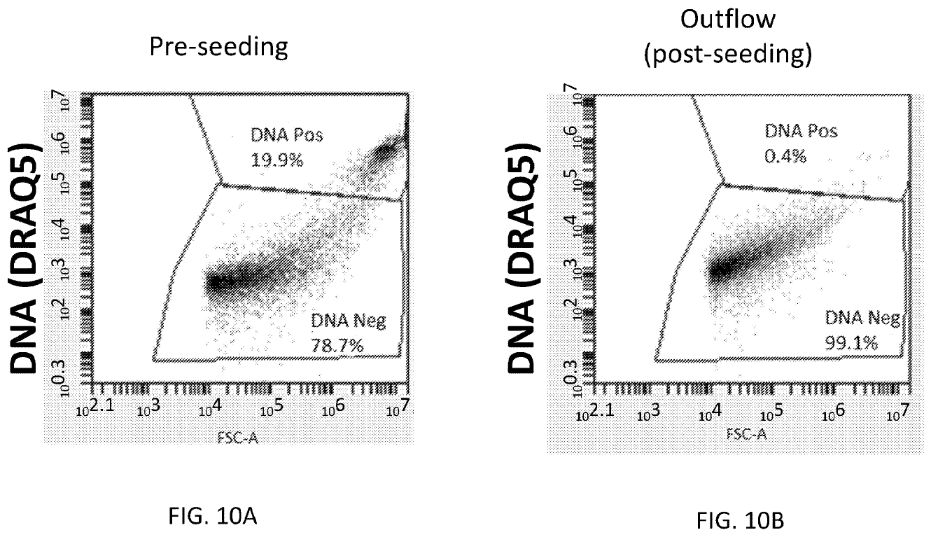

As noted above, in some embodiments, the membrane used in the present bioreactors can include pores sized to selectively capture, in the first channel, a biological source material capable of generating biological products and to permit the generated biological products to pass through the membrane into the second channel. For example, the flow cytometry plots in FIG. 10A and FIG. show a mixed population of large nucleated cells and platelet sized particles prior to seeding the cells in the bioreactor, and virtually only platelet sized particles in the outflow after seeding, indicating that all the nucleated cells, larger than the pore size, remain in the bioreactor, while smaller particles flow through the membrane pores.

In some embodiments, proper seeding can result in substantially all (e.g., about 99.9% or more) of the membrane pores capturing, and thus being filled with or blocked by, seeded biological source material. In such embodiments, equal flow can be supplied to the first and second channels such that the channels maintain a similar pressure drop per unit length, thereby permitting the flow resistor to produce a constant pressure drop across the membrane. In some embodiments, if only a portion of the pores are occupied (e.g., less than about 99.9% of membrane pores) at least some flow can pass through the membrane and thus the pressure drop across the membrane will vary along the length of the membrane. The flow through can create an imbalance in flow rates exiting the first and second channels. Therefore, in such embodiments, fluid may need to be added to the first channel recirculation loop so that the recirculated fluid does not become depleted, causing air to be pumped into the bioreactor.

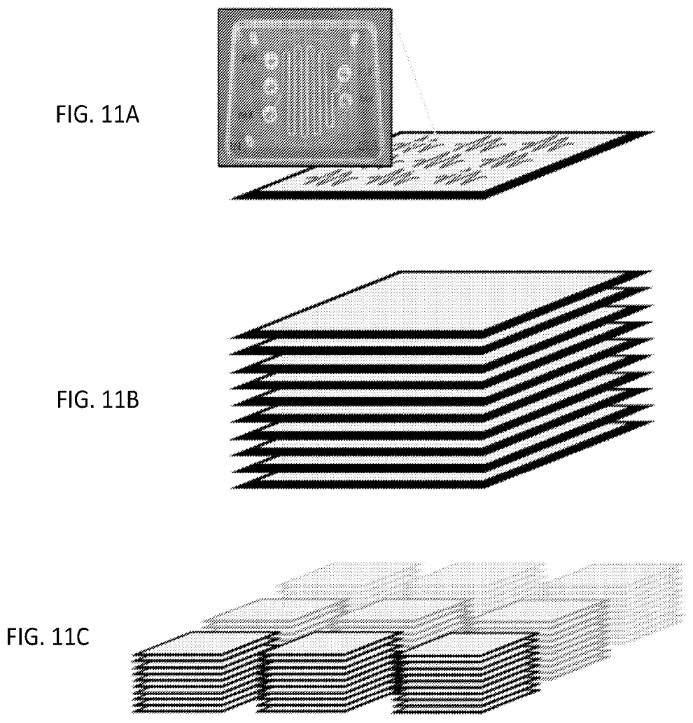

Referring now to FIGS. 11A-11C, production of biological products using bioreactors such as the bioreactors described herein with reference to FIGS. 3A-3F, 4A-4B, and 5 can be scaled according to user needs by implementation of a modular tablet bioreactor design wherein the first and second substrates are sized to include a plurality of bioreactor bodies. As shown in FIG. 11A, the first substrate of a tablet bioreactor configuration can include a plurality of first channels defined therein and the second substrate of a tablet configuration can include a plurality of opposing second channels defined therein in alignment with the plurality of first channels. As further shown in FIG. 11A, each of the bioreactor bodies formed within the modular tablet bioreactor can include first, second, and third inlets and first and second outlets as described above. The multiplexed bioreactor channels can be arranged in other configurations, such as radially aligned in a circular platform where multiple channels are fluidically connected or independently parallelized.

The individual bioreactor bodies formed within the tablet, in some embodiments, can be configured to operate in parallel or in series. For example, each bioreactor body (or multiple groups of bioreactor bodies) can be independently operated in parallel, wherein each bioreactor body or group of bioreactor bodies includes first and second pumps and first and second recirculation lines as described above with reference to FIG. 5. In some embodiments, for example, two or more of the bioreactor bodies can be connected in series such that, once seeded, a single pair of first and second pumps can be configured to provide flow through each of the series-connected bioreactor bodies. It will be appreciated in view of this disclosure that, in some embodiments, pump input flow rate can be independent of the number of bioreactor bodies in series, although larger reservoirs may be required to accommodate system volume.

Referring now to FIG. 11B, further scalability is contemplated by use of a stacked bioreactor including a plurality (e.g., 10 as shown) of modular tablet bioreactors arranged in a stack for increased production capacity. Referring now to FIG. 11C, still further scalability is contemplated by use of an industrial bioreactor including a plurality of the stacked bioreactors for still further increased production capacity.

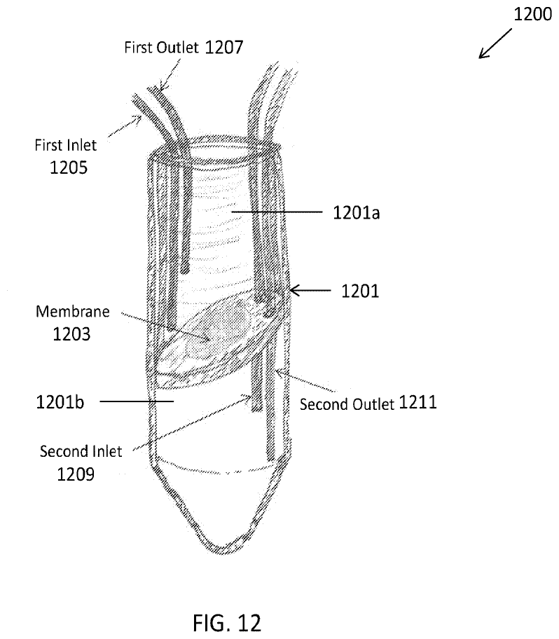

As shown in FIG. 12, in some embodiments, one or more recirculating bioreactors can each be provided with a single reservoir, rather than two separate reservoirs as depicted in FIGS. 5, 8, 9, and 10. As shown in FIG. 12, the bioreactor 1200 can include a reservoir 1201 having a first portion 1201a and a second portion 1201b divided by a membrane 1203. The reservoir 1201 can also include a first inlet 1205 and a first outlet 1207 for delivering and exiting flow from the first portion 1201a. The reservoir 1201 can also include a second inlet 1209 and a second outlet 1211, extending through the membrane 1203 for delivering and exiting flow from the second portion 1201b. In some embodiments, the first outlet 1207 is placed in the first portion 1201a and spaced apart from the membrane 1203. The first inlet 1205 is placed in the first portion 1201a proximate the membrane 1203 so it can collect biological source material that settles to a bottom of the first portion 1201a. The second inlet 1209 is placed in the second portion 1201b and spaced apart from a bottom of the second portion 1201b to prevent it from aspirating biological products that settle to the bottom of the second portion 1201b. The second outlet 1211 is placed proximate the bottom of the second chamber 1201b. When differences in flow rate between the first outlet 1207 and second outlet 1211 are generated (e.g., by a flow resistor as described above) or other asymmetries, the single reservoir 1201 can eliminate the need for addition of fluid to the first portion 1201a to sustain a recirculation loop (not shown) between the first outlet 1207 and the first inlet 1205. In particular, excess liquid is permitted to flow out of the second outlet 1211, be recirculated into the second portion 1201b via the second inlet 1209, and then flow upward through the membrane 1203 to further supply the first portion 1201a with fluid for recirculation between the first outlet 1207 and the first inlet 1205. This system 1200 also concentrates the biological products in the second portion 1201b for subsequent extraction and harvesting.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1.-41. (canceled)

42. A bioreactor comprising:
one or more bioreactor bodies, wherein at least one bioreactor body includes a first substrate and an opposing second substrate engaged with the first substrate;
a pathway extending through the bioreactor body and being formed by a first channel defined in the first substrate and an opposing second channel defined in the second substrate being opposite to the first channel, the first channel being configured to receive a biological source material capable of generating biological products and the second channel being in alignment with the first channel;
a first inlet for introducing a first fluid flow to the first channel;
a second inlet for introducing a second fluid flow to the second channel;
a third inlet for introducing the biological source material to the first channel;
a first outlet for permitting the first fluid flow to exit the first channel;
a second outlet for permitting the second fluid flow to exit the second channel;
a membrane disposed in the pathway between the first and second channels, the membrane including a plurality of pores, the pores being sized to selectively capture, in the first channel, a biological source material capable of generating biological products and to permit the generated biological products to be collected from the first channel or pass through the membrane into the second channel;
a first pump for recirculating the first fluid flow from the first outlet to the first inlet via a first recirculation line and through the first channel at a first flow rate;
a second pump for recirculating the second fluid flow from the second outlet to the second inlet via a second recirculation line and through the second channel at a second flow rate;
a third pump for delivering the biological source material to the first channel via the third inlet;
at least one valve positioned in fluid communication with each of the first inlet, the second inlet, the first outlet, and the second outlet for selectively permitting or preventing flow through at least one of the first channel, the second channel, the first recirculation line, and the second recirculation line; and
a flow controller configured to control the first pump and the second pump to independently adjust at least one of the first flow rate or the second flow rate through the first channel and the second channel, respectively, to maintain a shear rate in a range approximately between 10 sec-1 and 5000 sec-1 on the biological source material captured by the membrane and pressure through the membrane at desired rates to induce the biological source material to produce the biological products;
wherein a height of the first channel and a height of the second channel are varied along a length of the pathway to produce a uniform pressure drop across the membrane along the length of the pathway.

43. The bioreactor of claim 42, wherein the biological source material includes one or more of cells including stem cells and/or intermediate and/or final product of stem cell differentiation such as hemogenic endothelia, hematopoietic progenitor cells, megakaryocytes, endothelial cells, leukocytes, erythrocytes bone marrow cells, blood cells, lung cells, cells comprising basement membranes, and/or small molecules including CCL5, CXCL12, CXCL10, SDF-1, FGF-4, S1PR1, RGDS, Methylcellulose, and extracellular matrix proteins including collagen, fibrinectin, fibrinogen, laminin, Matrigel, Flt-3, TPO, VEGF, PLL, IL3, 6, 9, 1b, vitronectin, or combinations thereof.

44. The bioreactor of claim 43, wherein the biological products include one or more of products of the biological source material, components of the biological source material, or combinations thereof, optionally wherein the biological source material includes megakaryocytes and the biological products include one or more of preplatelets, proplatelets, platelets or their component products.

45. The bioreactor of claim 43, wherein at least one of the first fluid flow and the second fluid flow includes a fluid media including one or more biological substances including one or more of cell culture media, whole blood, plasma, platelet additive solutions, suspension media, saline, phosphate buffered saline, or combinations thereof.

46. The bioreactor of claim 42, wherein the plurality of pores of the membrane are further sized to prevent the biological source materials and biological products from passing through the membrane.

47. The bioreactor of claim 42, wherein the flow in at least one of the first channel or the second channel is one of peristaltic flow or laminar flow.

48. The bioreactor of claim 47, wherein the peristaltic flow is pulsatile with a physiologically relevant frequency between 40 and 120 pulses per minute.

49. The bioreactor of claim 48, wherein a shear rate generated at the membrane during the pulsatile peristaltic flow varies through a physiologically relevant range between 250 sec⁻¹ and 1800 sec⁻¹.

50. The bioreactor of claim 42, wherein the first substrate is bonded to the second substrate, optionally wherein the membrane is bonded between the first substrate and the second substrate.

51. The bioreactor of claim 42, wherein a height of the first channel and a height of the second channel are sized to produce a uniform shear at a surface of the membrane along the length of the pathway or a pressure through the membrane.

52. The bioreactor of claim 42, wherein a taper angle formed between a surface of each channel and the membrane is in a range approximately between 0 and 5 degrees.

53. The bioreactor of claim 42, wherein one or more of the first substrate, the second substrate and the membrane comprises one or more of thermoplastics, glass, polymethyl methacrylate (PMMA), polydimethylsiloxane (PDMS), polycarbonate (PC), cyclic olefin copolymer (COC), cyclic olefin polymer (COP), polyvinyl chloride (PVC), coated polystyrene, coated glass, silk, hydrogels, or combinations thereof.

54. The bioreactor of claim 42, wherein the plurality of pores are sized in a range approximately between 0.1 micrometers and 50 micrometers.

55. A method for generating biological products, the method comprising:
providing a bioreactor comprising:
at least one bioreactor body including a first substrate and an opposing second substrate engaged with the first substrate,
a pathway extending through the bioreactor body and being formed by a first channel defined in the first substrate and an opposing second channel defined in the second substrate, the second channel being in alignment with the first channel,
a membrane disposed in the pathway between the first and second channels, the membrane including a plurality of pores, the pores being sized to selectively capture, in the first channel, a biological source material capable of generating biological products and to permit the generated biological products to pass through the membrane into the second channel, wherein a height of the first channel and a height of the second channel are varied along a length of the pathway to produce a uniform pressure drop across the membrane along the length of the pathway;
a first pump for recirculating a first fluid flow from a first outlet to a first inlet via a first recirculation line and through the first channel at a first flow rate;
a second pump for recirculating a second fluid flow from a second outlet to a second inlet via a second recirculation line and through the second channel at a second flow rate;
at least one valve positioned in fluid communication with each of the first inlet, the second inlet, the first outlet, and the second outlet for permitting or preventing flow through at least one of the first channel, the second channel, the first recirculation line, and the second recirculation line; and
a flow controller configured to control the first pump and the second pump to independently adjust at least one of the first flow rate or the second flow rate through the first channel and the second channel, respectively;
introducing the biological source material to the first channel to seed the bioreactor;
introducing a first fluid flow to the first channel via a first inlet of the bioreactor at a predetermined first flow rate and a second fluid flow to the second channel via a second inlet of the bioreactor at a predetermined second flow rate to generate desired biological products;
recirculating the first fluid flow from the first outlet of the first channel to the first inlet via the first pump and recirculating the second fluid flow from a second outlet of the second channel to the second inlet via the second pump;
controlling, using the flow controller, the first pump and the second pump to independently adjust at least one of the first flow rate or the second flow rate through the first channel and the second channel, respectively, to maintain a shear rate in a range approximately between 10 sec-1 and 5000 sec-1 on the biological source material captured by the membrane and pressure through the membrane at desired rates to induce the biological source material to produce the biological products; and
harvesting the desired biological products from the bioreactor.

56. The method of claim 55, further comprising generating the biological source material from bone marrow, peripheral blood, umbilical cord blood, fetal liver, yolk sack, spleen, or pluripotent stem cells.

57. The method of claim 55, wherein the introducing the biological source material further comprises flowing a fluid containing the biological source material into the first channel, wherein distribution of the biological source material along the membrane is mediated by the flow of the fluid containing the biological source material.

58. The method of claim 57, wherein the biological source material, when selectively captured by one of the pores, blocks the pore, optionally wherein the selectively captured biological source material mediates fluid flow through the membrane.

59. The method of claim 57, further comprising:
monitoring a pressure drop across the membrane between the first channel and the second channel; and
determining, from the pressure drop, a density of the biological source material within the fluid containing the biological source material.

60. The method of claim 59, further comprising adjusting an introduced quantity of the fluid containing the biological source material in response to the determined density.

61. A bioreactor comprising:
one or more bioreactor bodies, wherein at least one bioreactor body includes a first substrate and an opposing second substrate engaged with the first substrate;
a pathway extending through the bioreactor body and being formed by a first channel defined in the first substrate and an opposing second channel defined in the second substrate, the second channel being in alignment with the first channel;
a first inlet for introducing a first fluid flow to the first channel;
a second inlet for introducing a second fluid flow to the second channel;
a third inlet for delivering a biological source material capable of generating biological products to the first channel;
a first outlet for permitting the first fluid flow to exit the first channel;
a second outlet for permitting the second fluid flow to exit the second channel;
a membrane disposed in the pathway between the first and second channels, the membrane including a plurality of pores, the pores being sized to selectively capture, in the first channel, the biological source material and to permit the generated biological products to be collected from the first channel or pass through the membrane into the second channel, wherein a height of the first channel and a height of the second channel are varied along a length of the pathway to produce a uniform pressure drop across the membrane along the length of the pathway;
a first pump for recirculating the first fluid flow from the first outlet to the first inlet via a first recirculation line and through the first channel at a first flow rate;
a second pump for recirculating the second fluid flow from the second outlet to the second inlet via a second recirculation line and through the second channel at a second flow rate;
at least one valve positioned in fluid communication with each of the first inlet, the second inlet, the first outlet, and the second outlet for permitting or preventing flow through at least one of the first channel, the second channel, the first recirculation line, and the second recirculation line; and
a flow controller configured to control the first pump and the second pump to independently adjust at least one of the first flow rate or the second flow rate through the first channel and the second channel, respectively, to maintain a shear rate on the biological source material captured by the membrane and pressure through the membrane at desired rates to induce the biological source material to produce the biological products.
